(19) United States
(12) Patent Application Publication
(10) Pub. No.: US 2004/0102718 A1
(43) Pub. Date: May 27, 2004

(54) Blood testing device

(76) Inventors: William M. Trudeau, Alta Loma, CA (US); James W. Boulgardies, El Segundo, CA (US)

Correspondence Address: CHRISTIE, PARKER & HALE, LLP, 350 WEST COLORADO BOULEVARD, SUITE 500, PASADENA, CA, 91105, US

(21) Appl. No.: 10/474,076

(22) PCT Filed: Aug. 24, 2001
(86) PCT No.: PCT/US01/26666

(51) Int. Cl. A61B 5/00

(52) U.S. Cl. 600/584

(57) ABSTRACT

A blood testing device (10) comprising a proximal end (12) and a distal end (14); an intermediate portion (20) between the proximal end (12) and the distal end (14); a pressure compensation system to regulate the pressure in the intermediate portion (20), and a distal end cap (60) at the distal end (14) comprising a first opening (62) configured to receive a test strip (104). The intermediate portion (20) comprises a proximal end (36) comprising one or more than one opening (44), a distal end (38) of the intermediate portion (20) and, between the proximal end (36) and the distal end (38), comprises a hollow, central chamber (40) surrounded by an outer wall (42).

### CROSS-REFERENCE TO RELATED APPLICATIONS

The present Application claims the benefit of U.S. Provisional Patent Application 60/281,957 titled “Unidirectional Self-sealing Blood Testing Apparatus,” filed Apr. 6, 2001; the contents of which are incorporated by reference in this disclosure in their entirety.

### BACKGROUND

The practice of medicine frequently involves testing blood for the presence or amount of one or more than one constituent, such as glucose, cardiac enzymes or red cells, or testing blood to determine the level of one or more than one parameter, such as pH. Such testing is often done while performing an intravascular access procedure by allowing blood from the distal end of an intravascular access apparatus to contact the end of a test strip, and then inserting the test strip into an appropriate test strip reader. This might be done, for example, by allowing blood to leak out of a catheter that has accessed the vascular structure, prior to attaching the intravenous fluid administration tubing, onto the test strip. Another method of gaining access to a patient's blood is to force the blood out of the vascular access device onto the test strip.

Frequently, however, intravascular access procedures are performed by emergency medical personnel or by paramedics under adverse environmental conditions, such as inclement weather. Further, intravascular access procedures are often performed by emergency medical personnel or by paramedics in moving vehicles. These conditions increase the possibility of inaccurate blood test results due to test strip contamination, and increase the risk of exposure of the emergency medical personnel or paramedics to pathogens present in the blood being tested.

Therefore, it would be useful to have a device for testing blood conditions that decreases the possibility of inaccurate blood test results due to test strip contamination. Further, it would be useful to have a device for testing blood conditions that decreases the risk of exposure of healthcare providers to pathogens present in the blood.

### SUMMARY

In one embodiment, there is provided a blood testing device for testing blood for the presence or for the amount of one or more than one constituent, or for testing blood to determine the level of one or more than one parameter, or for testing blood for a combination of the preceding.

The blood testing device comprises a proximal end of the blood testing device and a distal end of the blood testing device; an intermediate portion between the proximal end of the blood testing device and the distal end of the blood testing device; and a pressure compensation system to regulate the pressure in the intermediate portion; and the intermediate portion comprises a proximal end of the intermediate portion comprising one or more than one opening, a distal end of the intermediate portion and, between the proximal end of the intermediate portion and the distal end of the intermediate portion, comprises a hollow, central chamber surrounded by an outer wall.

In one preferred embodiment, the pressure compensation system comprises a distal portion between the proximal end of the blood testing device and the distal end of the blood testing device, an inner membrane separating the intermediate portion from the distal portion; and the distal portion comprises a proximal end of the distal portion and a distal end of the distal portion and, between the proximal end of the distal portion and the distal end of the distal portion, comprises a hollow, central chamber surrounded by an outer wall.

In another preferred embodiment, the pressure compensation system comprises a pressure compensation valve, which can be a duck bill valve.

In one embodiment, the blood testing device further comprises a distal end cap at the distal end of the blood testing device comprising a first opening configured to receive a test strip. Embodiments are described using an end cap that is a semi-permeable member, and a non-permeable member.

In another embodiment, the outer wall of the intermediate portion is generally cylindrical.

In another embodiment, the outer wall of the distal portion is generally cylindrical.

In another preferred embodiment, the blood testing device further comprises a proximal portion between the proximal end of the blood testing device and the proximal end of the intermediate portion. In a further embodiment, the proximal portion comprises a hollow, central chamber surrounded by an outer wall, and the proximal portion is configured to mate with a blood containing apparatus. In a further embodiment, the blood containing apparatus is selected from the group consisting of a syringe and the introducer part of an intravascular access apparatus. In a further embodiment, the outer wall of the proximal portion is generally cylindrical.

In another preferred embodiment, the hollow, central chamber of the intermediate portion and the hollow, central chamber of the distal portion comprise generally rectangular inferior parts that communicate with each other and that are configured to accept a test strip. In a further particularly preferred embodiment, the hollow, central chamber of the distal portion further comprises one or more than one ridge axially along the proximal to distal axis.

In another embodiment, the outer wall of the intermediate portion is substantially transparent.

In another embodiment, the distal end cap is an integral part of the distal portion of the blood testing device.

In another embodiment, the distal end cap is removably affixed to the distal portion of the blood testing device.

In another embodiment, the distal end cap further comprises one or more than one second opening to allow the venting of air externally out of the distal portion of the blood testing device.

In another preferred embodiment, the inner membrane is a semipermeable member.

In another embodiment, the inner membrane comprises one or more than one vent.

In another embodiment, the outer wall of the intermediate portion comprises one or more than one vent.

In another embodiment, the inner membrane comprises an opening configured to allow passage of a test strip from the distal portion of the blood testing device into the intermediate portion of the blood testing device.

In another embodiment, the intermediate portion is integrally affixed to an introducer portion of an intravascular access apparatus. In a further embodiment the intermediate portion is configured to affix to an introducer portion of an intravascular access apparatus.

In another preferred embodiment, the distal end of the blood testing device is integrally affixed to a blood collection apparatus. In a further embodiment, the distal end of the blood testing device is configured to affix to a blood collection apparatus. In one embodiment, the blood collection apparatus may be a syringe.

In another preferred embodiment, the intermediate portion is either integrally affixed or configured to affix to an introducer portion of an intravascular access apparatus, and the distal end is either integrally affixed or configured to affix to a blood collection apparatus. In one embodiment, the blood collection apparatus may be a syringe.

In another embodiment, the present invention is a blood testing apparatus comprising a plurality of interconnected blood testing devices according to the present invention.

In another embodiment, the present invention is a method for testing blood for the presence or for the amount of one or more than one constituent, or for testing blood to determine the level of one or more than one parameter, or for testing blood for a combination of the preceding. The method comprises the steps of, first, providing a blood testing device according to the present invention, a test strip comprising a proximal end and a distal end, a test strip reader configured to accept the distal end of the test strip and a blood containing apparatus. Next, the blood testing device is affixed to the blood containing apparatus and blood present in the blood containing apparatus is allowed to enter into the intermediate portion of the blood testing device. Then, the distal end of the test strip is inserted into the test strip reader and the proximal end of the test strip is inserted into the intermediate portion of the blood testing device. Next, an operator uses information provided by the test strip reader to determine the presence or the amount of one or more than one constituent of the blood, or to determine the level of one or more than one parameter, or to determine a combination of the preceding. In one embodiment, the blood testing apparatus is selected from the group consisting of an intravascular access apparatus and a syringe. The blood testing device and blood containing apparatus can be affixed integrally.

In another embodiment, the method further comprises selecting a patient whose blood is to be tested and intravascularly accessing the patient's blood with the blood containing apparatus. In another embodiment, the method further comprises the step of discarding the blood testing device.

### FIGURES

These and other features, aspects and advantages of the present invention will become better understood with regard to the following description, appended claims, and accompanying figures where:

FIG. 1 is a lateral perspective view of a blood testing device according to one embodiment of the present invention;

FIG. 2 is a cross-sectional, lateral perspective view of the blood testing device shown in FIG. 1;

FIG. 3 is a lateral perspective view of a blood testing device shown in FIG. 1 attached to a standard intravascular access apparatus;

FIG. 4 is a lateral perspective view of a semipermeable membrane forming part of the blood testing device of FIG. 1;

FIG. 5 is a lateral perspective view of a blood testing device according to another embodiment of the present invention;

FIG. 6 is a lateral perspective view of a blood testing device according to another embodiment of the present invention;

FIG. 7 is a lateral perspective view of a blood testing device according to another embodiment of the present invention;

FIG. 8 is a lateral perspective view of a blood testing apparatus comprising a plurality of interconnected blood testing devices according to the present invention;

FIG. 9 is a cross-sectional, lateral perspective view of the blood testing device shown in FIG. 1 according to another embodiment of the present invention;

FIG. 10A is a cross-sectional view of a duck bill valve forming part of the blood testing device of FIG. 6, before insertion of a test strip;

FIG. 10B is a cross-sectional view of a duck bill valve forming part of the blood testing device of FIG. 6, after insertion of a test strip;

FIG. 11 is a cross-section and cut-away lateral view of a blood testing device according to another embodiment of the present invention;

FIG. 12 is a cross-section and cut-away lateral view of a blood testing device according to another embodiment of the present invention;

FIG. 13 is a cross-section and cut-away lateral view of a blood testing device according to another embodiment of the present invention;

FIG. 14 is a cross-section and cut-away lateral view of a blood testing device according to another embodiment of the present invention;

FIG. 15 is a cross-section and cut-away lateral view of a blood testing device according to another embodiment of the present invention;

FIG. 16 is a cross-section and cut-away lateral view of a blood testing device according to another embodiment of the present invention; and

FIG. 17 is a cross-section and cut-away lateral view of a blood testing device according to another embodiment of the present invention.

### DESCRIPTION

According to one embodiment of the present invention, there is provided a blood testing device for testing blood for the presence or for the amount of one or more than one constituent, such as glucose, cardiac enzymes or red cells, or for testing blood to determine the level of one or more than one parameter, such as pH, or for testing blood for a combination of the preceding. The blood testing device decreases the possibility of inaccurate blood test results due to test strip contamination, and decreases the risk of exposing an operator to pathogens present in the blood.

According to another embodiment of the present invention, there is provided a method for testing blood for the presence or for the amount of one or more than one constituent, such as glucose, cardiac enzymes or red cells, or for testing blood to determine the level of one or more than one parameter, such as pH, or for testing blood for a combination of the preceding. The method comprises providing a testing means that decreases the possibility of inaccurate blood test results due to test strip contamination, and decreases the risk of exposing an operator to pathogens present in the blood. These embodiments will be disclosed in greater detail in the following disclosure.

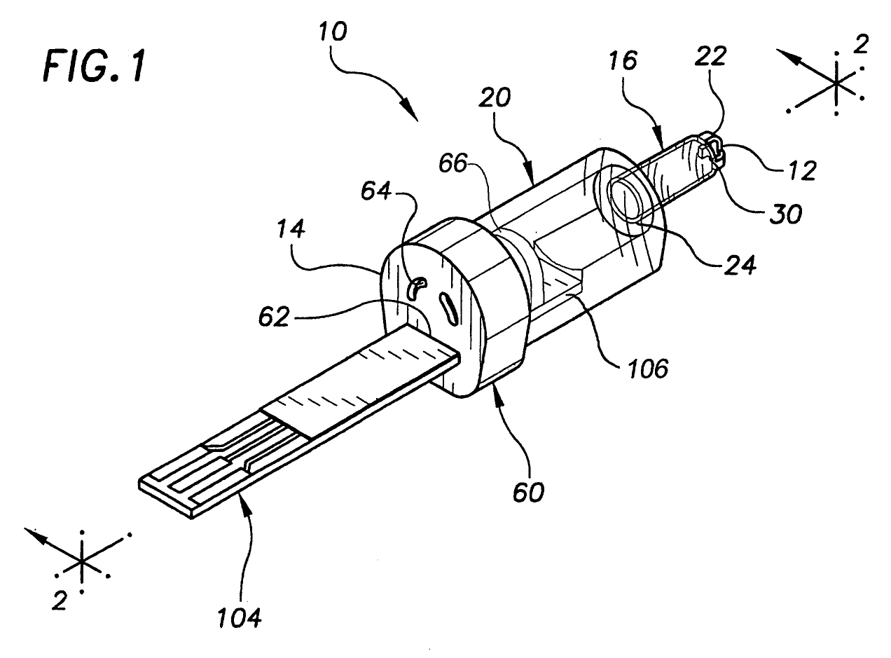

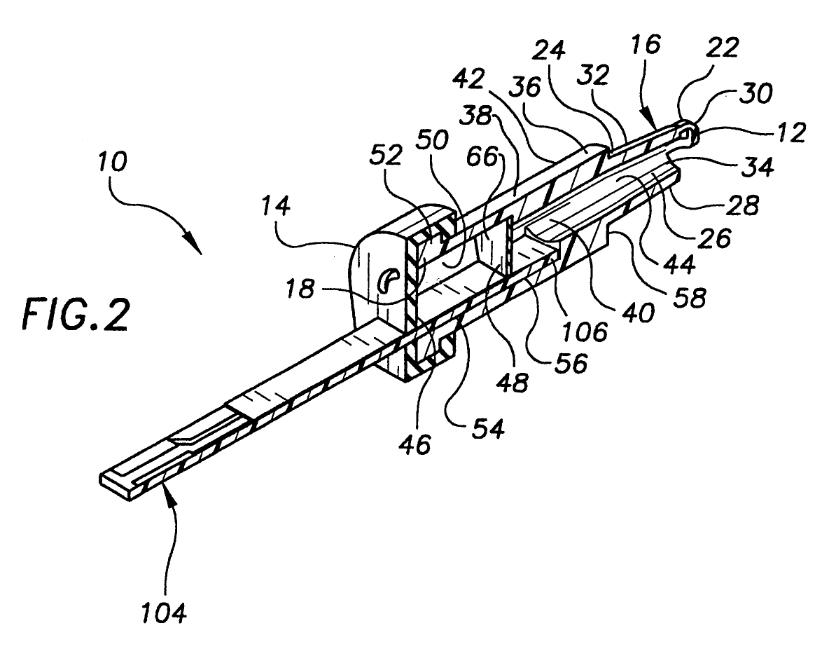

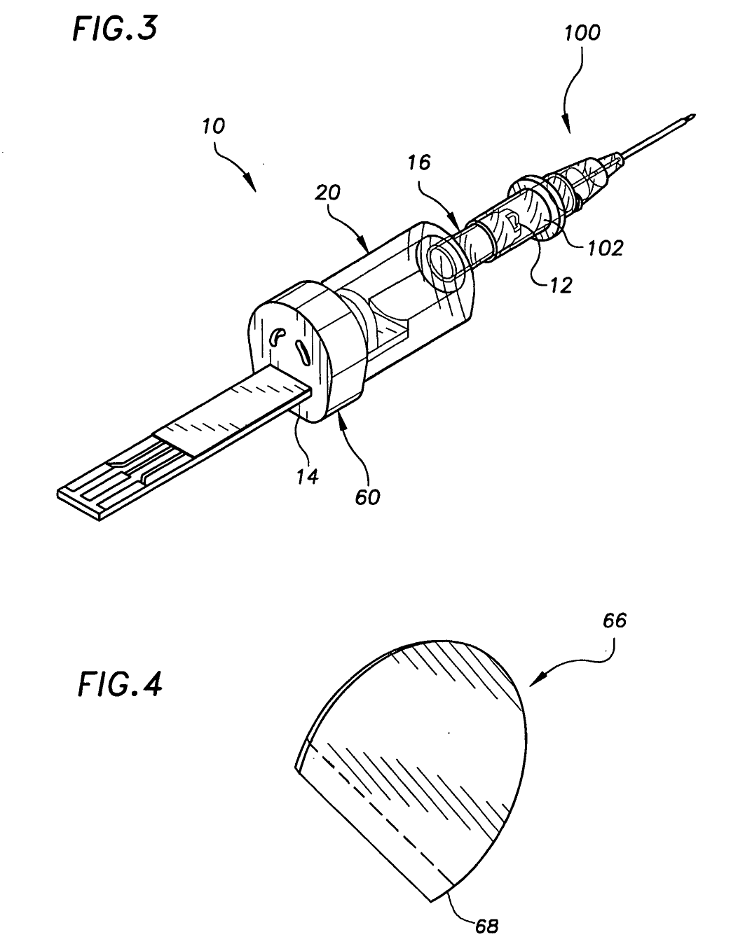

Referring now to FIG. 1, FIG. 2 and FIG. 3, there are shown, respectively, a lateral perspective view of a blood testing device 10 according to one embodiment of the present invention; a cross-sectional, lateral perspective view of a blood testing device 10 shown in FIG. 1; and a lateral perspective view of a blood testing device 10 shown in FIG. 1 combined with a intravascular access apparatus 100. As can be seen, in this embodiment the blood testing device 10 comprises a proximal end 12 and a distal end 14. Between the proximal end 12 and the distal end 14, the blood testing device 10 further comprises a proximal portion 16, a distal portion 18, and an intermediate portion 20 between the proximal portion and the distal portion.

The proximal portion 16 comprises a proximal end 22 and a distal end 24 and, between the proximal end 22 and the distal end 24, further comprises a hollow, central chamber 26 surrounded by an outer wall 28 having a long axis in the proximal to distal direction. The outer wall 28 of the proximal portion 16 is configured to mate with the distal end of a blood containing apparatus, such as a syringe (not shown) or the introducer part 102 of an intravascular access apparatus 100, as shown in FIG. 3. Preferably, the outer wall 28 of the proximal portion 16 is configured to mate with the distal end of the introducer part of a standard intravascular access apparatus, such as an Introcan® Safety™ IV Catheter (B. Braun Medical Inc., Bethlehem, Pa. US) or other standard intravascular access apparatus, and, therefore, the outer wall 28 of the proximal portion 16 will vary in configuration depending on the intravascular access apparatus that will be used in conjunction with the blood testing device 10 of the present invention, as will be understood by those with skill in the art with reference to this disclosure.

In a preferred embodiment, as shown in FIG. 1 and FIG. 2, the outer wall 28 of the proximal portion 16 comprises a generally tapered proximal-most part 30 and a generally cylindrical distal-most part 32. The proximal-most part 30 of the outer wall 28 preferably further comprises one or more than one opening 34 allowing the hollow, central chamber 26 to communicate externally, or when combined with a blood containing apparatus, to communicate with the blood containing part of the blood containing apparatus. The one or more than one opening 34 is configured to allow the passage of blood that is present in the blood containing apparatus into the hollow, central chamber 26 of the proximal portion 16.

The intermediate portion 20 comprises a proximal end 36 and a distal end 38 and, between the proximal end 36 and the distal end 38, comprises a hollow, central chamber 40 surrounded by an outer wall 42 having a long axis in the proximal to distal direction. Preferably, the outer wall 42 of the intermediate portion 20 is generally cylindrical, though other configurations are also suitable, as will be understood by those with skill in the art with reference to this disclosure. The proximal end 36 of the intermediate portion 20 comprises one or more than one opening 44 allowing the hollow, central chamber 40 of the intermediate portion 20 to communicate with the hollow, central chamber 26 of the proximal portion 16.

The distal portion 18 comprises a proximal end 48 and a distal end 46 and, between the proximal end 48 and the distal end 46, comprises a hollow, central chamber 50 surrounded by an outer wall 52 with a central axis in the proximal to distal direction. Preferably, the outer wall 52 of the distal portion 18 is generally cylindrical, though other configurations are also suitable, as will be understood by those with skill in the art with reference to this disclosure.

The hollow, central chamber 50 has a sufficient size and suitable shape to receive a test strip 104 or other test apparatus inserted into the hollow, central chamber 50 from the distal end 14 of the blood testing device 10 toward the proximal end 12. As used in this disclosure, the term “test strip” is understood to include other test apparatuses that are not in linear strip form but are suitable for use with the blood testing device 10 of the present invention. Therefore, the configuration of the hollow, central chamber 50 of the distal portion 18 will depend upon the configuration of the test strip to be used, as will be understood by those with skill in the art with reference to this disclosure. Suitable test strips include chemical test strips, such as One Touch® FastTake® (LifeScan, Inc., Milpitas, Calif. US) and electrochemical test strips, such as Precision QID (MediSense, Bedford, Mass. US).

In a particularly preferred embodiment, as shown in FIG. 1, FIG. 2 and FIG. 3, the hollow, central chamber 50 of the distal portion 18, and the hollow, central chamber 40 of the intermediate portion 20, comprises generally rectangular (when viewed from an inferior direction), inferior part 54 and inferior part 56, respectively, as shown, that communicate with each other and that are configured to accept a test strip 104. The proximal end 58 of the outer wall of the inferior part 56 of the intermediate portion 20 is closed.

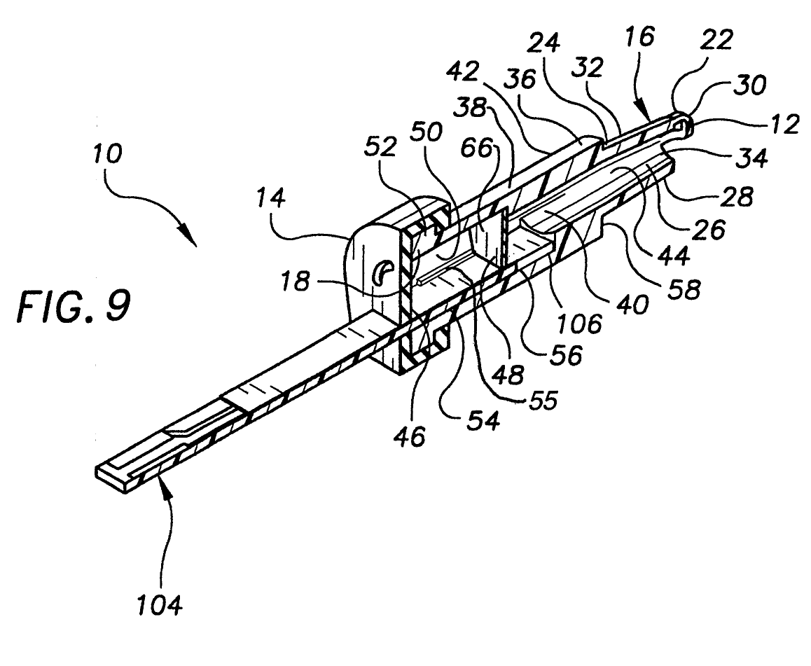

In another preferred embodiment, as shown in FIG. 9, inferior part 54 of central chamber 50 of distal portion 18 further comprises one or more than one ridge 55 having a long axis in the proximal to distal direction. Ridge 55 acts a guide for test strip 104 in inferior part 54, and also helps to prevent blood from gaining access to the superior space in hollow, central chamber 50. Ridge 55 may extend for part or all of the length of central chamber 50, and may extend for part or all of the width of central chamber 50.

The outer wall 28 of the proximal portion 16, the outer wall 42 of the intermediate portion 20 and the outer wall 52 of the distal portion 18 preferably comprise a plastic material such as polycarbonate, that does not affect the constituents or parameters of blood being tested, though other materials are suitable as will be understood by those with skill in the art with reference to this disclosure. The material used is preferably inexpensive, as the blood testing device 10 is intended to be discarded after a single use, and is capable of being manufactured into the proper shape according to standard techniques known to those with skill in the art, such as by injection molding. Further preferably, the material is either substantially transparent or at least sufficiently transparent to allow visual confirmation that blood is present in the hollow, central chamber 40 of the intermediate portion 20.

The distal end 14 of the blood testing device 10 further comprises a distal end cap 60. In one embodiment (not shown), the distal end cap 60 is an integral part of the distal portion 18 of the blood testing device 10, and of the same or of a different material as the outer wall 52 of the distal portion 18. In a preferred embodiment, as shown in FIG. 1, FIG. 2 and FIG. 3, the distal end cap 60 is a separate part, removable from the distal portion 18, and comprises a resilient material such as natural or synthetic rubber that, when placed over the distal portion 18 of the blood testing device 10, tends to remain attached to the distal portion 18 of the blood testing device 10 due to the resilient nature of the material.

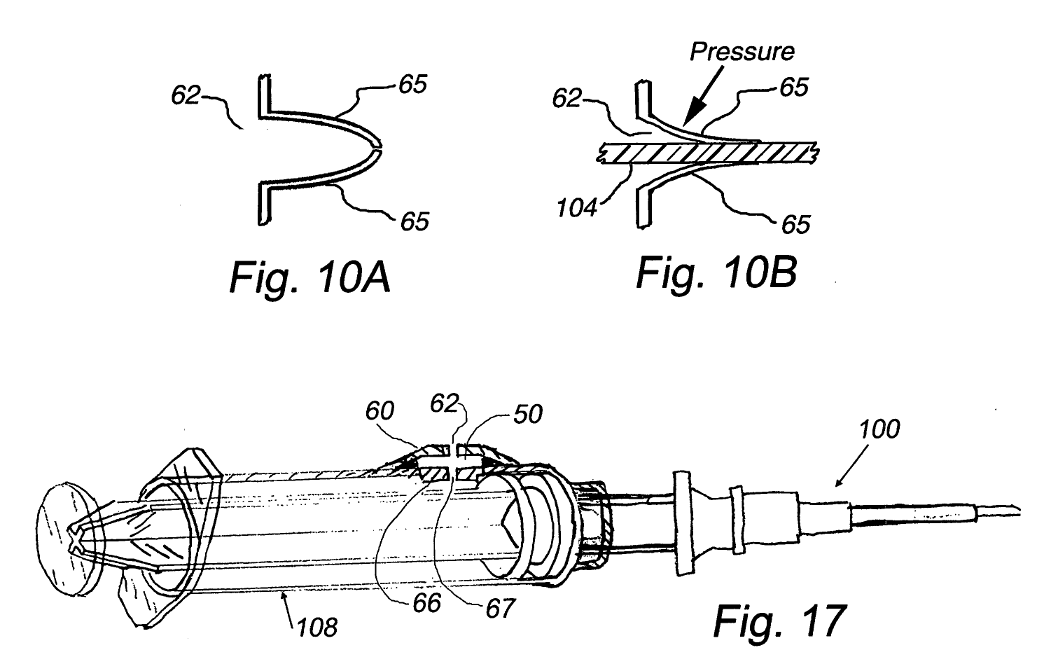

In one embodiment, the distal end cap 60 further comprises at least a first opening 62 configured to snugly receive a test strip 104 inserted into the hollow, central chamber 50 of distal portion 18 of the blood testing device 10. Therefore, the configuration of the first opening 62 in the distal end cap 60 will depend upon the configuration of the test strip to be used, as will be understood by those with skill in the art with reference to this disclosure. In a preferred embodiment, the first opening 62 is generally slit-like, as shown in FIG. 1, FIG. 2 and FIG. 3. The first opening 62 can comprise a simple opening into the hollow, central chamber 50 of the distal portion 18, as shown, or can comprise a valve, such as a duck bill valve 65 as shown in FIG. 10A and FIG. 10B. In a particularly preferred embodiment, as shown in FIG. 1, FIG. 2 and FIG. 3, the distal portion 18 and the intermediate portion 20 each comprises the generally rectangular, inferior part 54 and 56 and the long axis of the first opening 62 is a slit that is aligned perpendicular to the direction of insertion of the long axis of the test strip 104 into the distal portion 18.

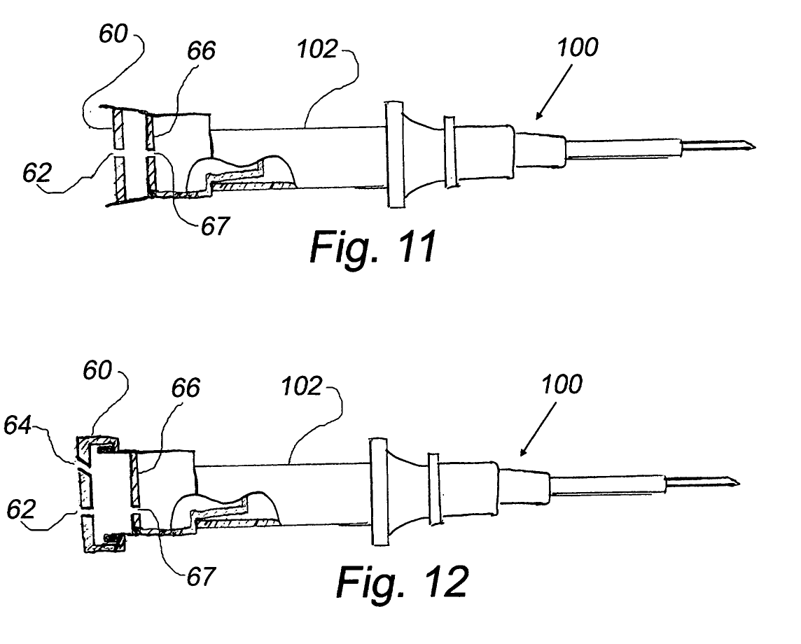

The distal end cap 60 can comprise a semipermeable membrane, as shown in FIG. 11, that allows the selective passage of air out of the distal portion 18, while retaining the fluid and cellular components of blood within the distal portion 18. The semipermeable membrane can comprise any suitable material, such as Porex T3 #X-7744, (Porex Medical Products Group, Fairburn, Ga. US) or Type TTTP Isopore Membrane (Millipore Corporation, Bedford, Mass. US), though other material is also suitable as will be understood by those with skill in the art with reference to this disclosure.

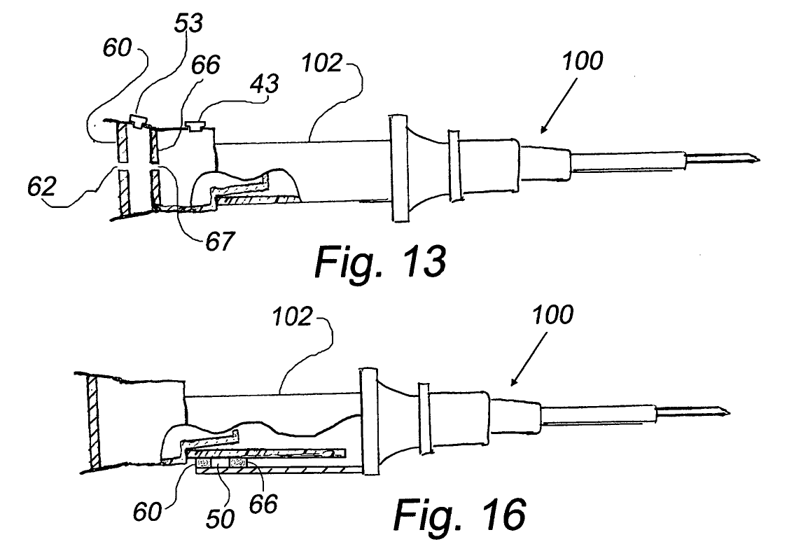

Additionally preferably, and particularly when the distal end cap 60 does not comprise a semipermeable membrane, the distal end cap 60 comprises a vent of one or more than one second opening 64 to allow the venting of air externally out of the distal portion 18 of the blood testing device 10. The second opening 64 can be any suitable configuration, such as generally slit-like, as shown in FIG. 1, as will be understood by those with skill in the art with reference to this disclosure. Alternatively, or additionally, as shown in FIG. 13, the outer wall 52 of the distal portion 18 can comprise one or more than one vent 53.

In one preferred embodiment, the blood testing device 10 further comprises an inner membrane 66 separating the distal end 38 of the hollow, central chamber 40 of the intermediate portion 20 from the proximal end 48 of the hollow, central chamber 50 of the distal portion 18. In a preferred embodiment, the inner membrane 66 is a semipermeable member. In another embodiment, the inner membrane 66 is not permeable but comprises one or more than one vent (not shown). Alternately, or additionally, as shown in FIG. 13 and FIG. 15, the outer wall 42 of the intermediate portion 20 can comprise one or more than one vent 43.

The inner membrane 66 comprises any suitable material, such as rubber or Porex T3 #X-7744, (Porex Medical Products Group, Fairburn, Ga. US), and can additionally be treated to be hydrophobic, as will be understood by those with skill in the art with reference to this disclosure.

Referring now to FIG. 4, there is shown a lateral perspective view of one embodiment of an inner membrane 66 forming part of the embodiment of the blood testing device 10 shown in FIG. 1, FIG. 2 and FIG. 3. The inner membrane 66 allows the selective passage of air through the inner membrane 66 between the distal end 38 of the hollow, central chamber 40 of the intermediate portion 20 and the proximal end 48 of the hollow, central chamber 50 of the distal portion 18, while preventing the passage of the fluid and cellular components of blood through the inner membrane 66.

The inner membrane 66 is further configured to allow passage of the test strip 104 from the distal portion 18 of the blood testing device 10 into the intermediate portion 20 of the blood testing device 10. For example, as shown in FIG. 12, the inner membrane 66 can comprise a slit 67 aligned with the first opening 62 in the distal end cap 60. In a further embodiment, the inner membrane 66 can comprise a perforation (not shown) aligned with the first opening 62 in the distal end cap 60. In that embodiment the perforation can be constructed so that it is breached by the insertion of test strip 104.

In a preferred embodiment, as shown in FIG. 1, FIG. 2 and FIG. 3, the intermediate portion 20 and the distal portion 18 each comprise the generally rectangular, inferior part 54 and 56 and the first opening 62 is a slit that is aligned perpendicular to the direction of insertion of the long axis of the test strip 104 into the distal portion 18, and the inner membrane 66 has an inferior extension 68 that further divides the inferior part 54 of the distal portion 18 from the inferior part 56 of the intermediate portion 20.

When a test strip is inserted through the distal end cap 60 and into the hollow, central chamber 50 of the inferior part 54 of the distal portion 18, the inferior extension 68 of the inner membrane 66 is displaced proximally, permitting the test strip to be further inserted into inferior part 56 of the intermediate portion 20, and allowing blood in the hollow, central chamber 40 of the intermediate portion 20 to contact the proximal end 106 of the test strip 104.

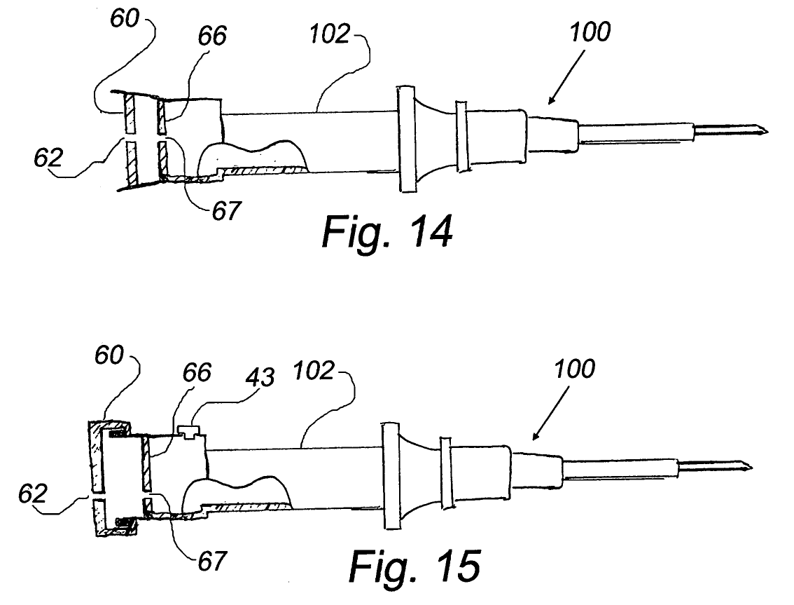

Referring now to FIG. 5, FIG. 14, and FIG. 15, there is shown lateral perspective views of a blood testing device 10 according to another embodiment of the present invention. As can be seen, in these embodiments, the blood testing device 10 is manufactured without a proximal portion 16 and with the intermediate portion 20 integrally affixed to the distal end of the introducer part 102 of a standard intravascular access apparatus 100. In these embodiments, the blood testing device 10 and introducer part 102 of an intravascular access apparatus 100 are inserted intravascularly and are removed as a unit, as will be understood by those with skill in the art with reference to this disclosure.

As will be understood by those with skill in the art with reference to this disclosure, as the test strip is inserted through the distal end cap 60 and into the hollow, central chamber 50, the pressure within central chamber 50 will increase. If no pressure compensation system has been included in the blood testing device for amelioration of this pressure increase, depending upon the nature of the material chosen for the inner membrane 66, a corresponding increase in the pressure within the central chamber 40 of the intermediate portion 20 may result. If no pressure compensation system has been included for equalization of pressure within central chamber 40, a small amount of blood may be expelled through the one or more than one opening 34 of the proximal-most part 30 of outer wall 28 of proximal portion 16. This may be undesirable to the health care worker inserting the test strip. As the test strip is further inserted into inferior part 56 of the intermediate portion 20, an additional increase in pressure within central chamber 40 will occur, increasing the likelihood of escape of blood through opening 34.

A preferred embodiment of a pressure compensation system is as shown in FIG. 1, FIG. 2, and FIG. 4, in which inner membrane 66 is a semipermeable member, comprising an inferior extension 68, and distal end cap 60 comprises a resilient material such as natural or synthetic rubber, and a vent of one or more than one second opening 64. As test strip 104 is inserted into blood testing device 10, this embodiment of the pressure compensation system allows pressure to equalize, resulting in little escape of blood from the intermediate portion into the distal portion, and no escape of blood from the blood testing device 10. Additionally preferably, central chamber 50 of distal portion 18 comprises one or more than one ridge 55 reducing the amount of blood that enters central chamber 50.

Another embodiment of a pressure compensation system, as shown in FIG. 11, comprises a distal end cap 60 comprising a semipermeable membrane that allows the selective passage of air out of the distal portion 18, together with inner membrane 66 being a semipermeable member, comprising an inferior extension 68. In this embodiment, second opening 64 is not required.

Another embodiment of a pressure compensation system comprises a distal end cap 60 comprising an integral part of the distal portion 18 of the blood testing device 10, and of the same or of a different material as the outer wall 52 of the distal portion 18, together with inner membrane 66 being a semipermeable member, comprising an inferior extension 68. In this embodiment, second opening 64 is located either on the distal end cap, or one or more than one vent 53 is located on outer wall 52.

Another embodiment of a pressure compensation system comprises an inner membrane 66 which is not permeable but comprises one or more than one vent (not shown). Alternately, or additionally, the outer wall 42 of the intermediate portion 20 can comprise one or more than one vent 43. In this embodiment, distal end cap 60 may optionally comprise (I) a resilient material such as natural or synthetic rubber, and a vent of one or more than one second opening 64 in the end cap, or the outer wall 52 of distal portion 18 can comprise one or more than one vent 53, (ii) a semipermeable membrane that allows the selective passage of air out of the distal portion 18, or (iii) an integral part of the distal portion 18 of the blood testing device 10, and of the same or of a different material as the outer wall 52 of the distal portion 18, and one or more than one second opening 64 in the end cap, or the outer wall 52 of distal portion 18 can comprise one or more than one vent 53.

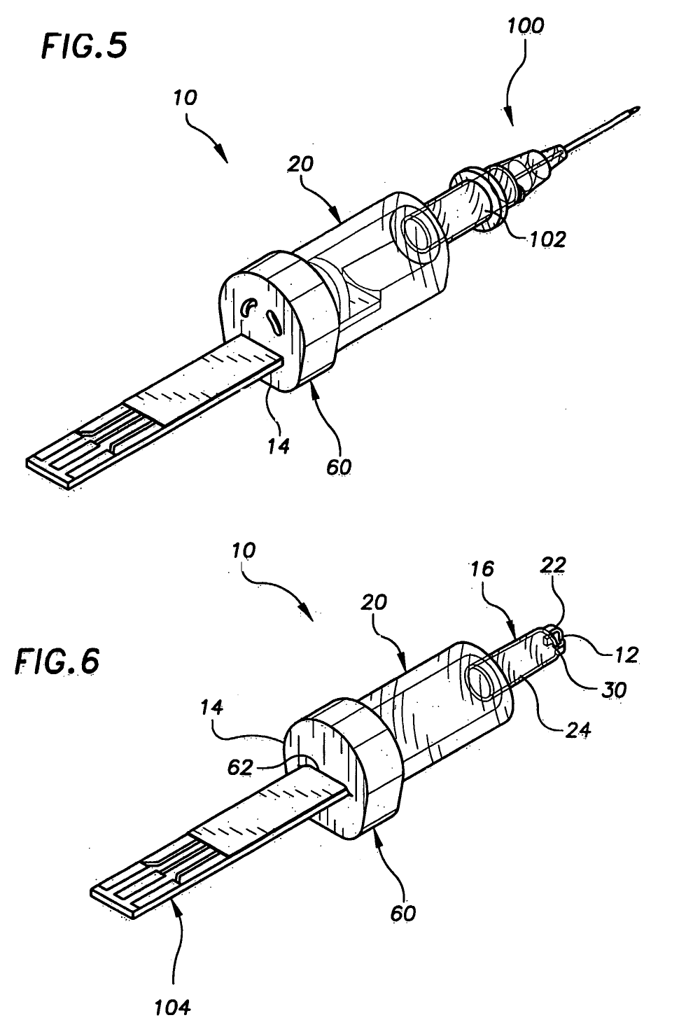

It is also possible to ameliorate the pressure increase caused by the insertion of the tests strip 104 through a pressure compensation valve. Referring now to FIG. 6, there is shown a lateral perspective view of a blood testing device 10 according to another embodiment of the present invention. As can be seen, in this embodiment, the blood testing device 10 comprises a distal end cap 60 where the first opening 62 comprises a duck bill valve 65, shown in cross-section in FIG. 10A before a test strip 104 is inserted, and shown in cross-section in FIG. 10B after a test strip 104 is inserted, the sides of which adjoin and seal together when no test strip 104 is present and which separate upon insertion of a test strip to permit insertion of the test strip into the hollow, central chamber 40 of the intermediate portion 20. In this embodiment of a pressure compensation system, the equalization of pressure is accomplished by the collapse of the structure of the duck bill valve, as is shown in FIG. 10B after the test strip 104 is inserted, and as will be understood by those with skill in the art with reference to this disclosure. Accordingly, in this embodiment opening 64 in end cap 60 and inner membrane 66 are not needed.

Having thus described several embodiments of a pressure compensation system, it will now be understood by those with skill in the art with reference to this disclosure that a pressure compensation system may be constructed with various combinations of the described components, as further shown in FIGS. 11 through 17.

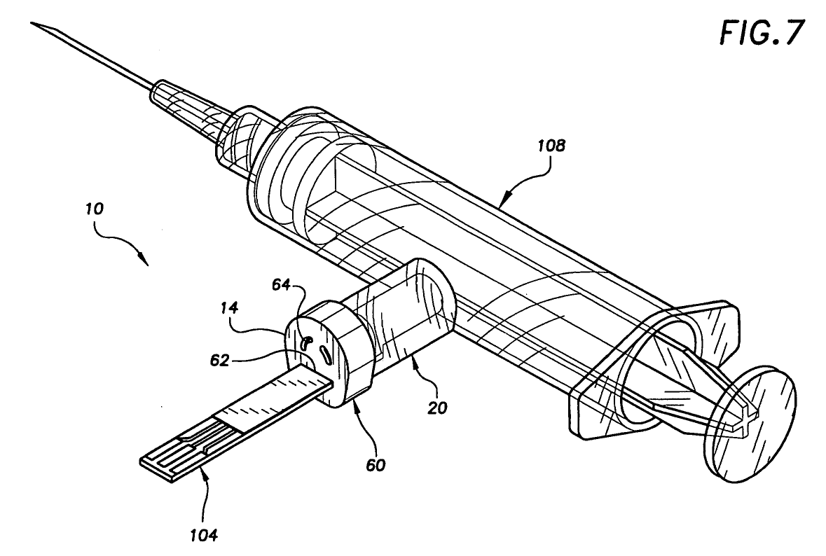

Referring now to FIG. 7 and FIG. 17, there is shown a lateral perspective view of a blood testing device 10 according to two other embodiments of the present invention. As can be seen, in these embodiments, the blood testing device 10 is manufactured integrally affixed to the side of a syringe 108 by connecting either the intermediate portion 20 of a blood testing device 10, as disclosed above, directly to the side of the syringe 108, as in FIG. 7, or the inner membrane 66 directly to the side of the syringe 108, as in FIG. 17, the interior of syringe 108 thus becoming the intermediate portion 20. Other blood collection devices could be used, as will be understood by those with skill in the art with reference to this disclosure.

It would also be possible to use a pressure compensation valve in either embodiment, and eliminate the inner membrane. In these embodiments, the syringe 108 is used to draw blood from a blood source and the blood testing device 10 is used to test the blood within the syringe 108 without having too expel the blood from the syringe 108. The blood testing device 10 can also be affixed to the distal end of the plunger of a syringe 108 where the plunger has a central channel in communication with the intermediate portion 20 of the blood testing device 10 (not shown).

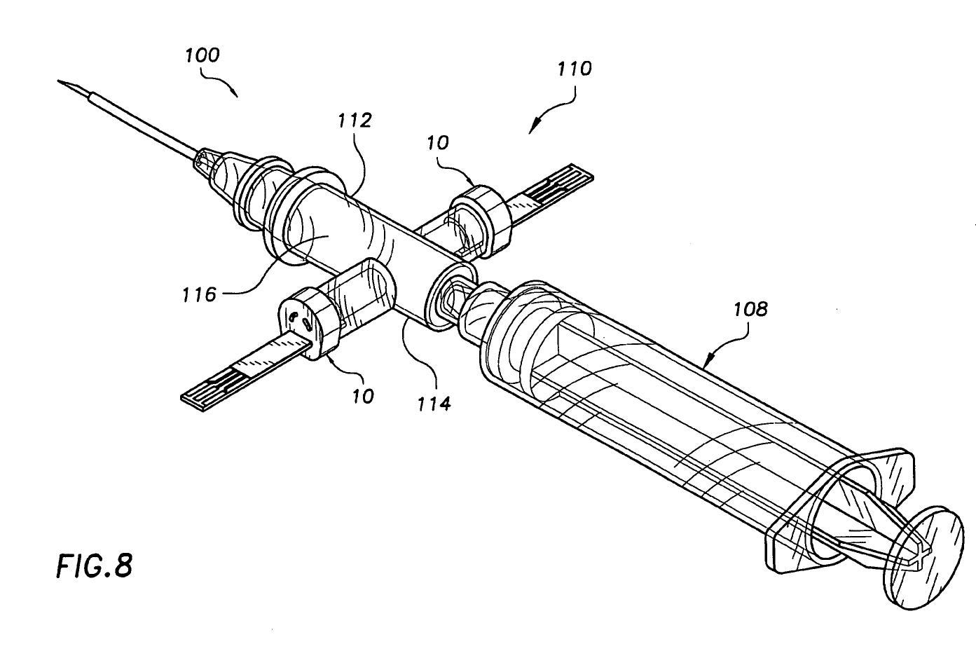

In another embodiment, the present invention includes a blood testing apparatus 110 comprising a plurality of interconnected blood testing devices 10 according to the present invention, to allow multiple test strips to be read from blood in the same blood containing apparatus 110. Referring now to FIG. 8, there is shown a lateral perspective view of such a blood testing apparatus 110. As can be seen, in this embodiment, the blood testing apparatus 110 comprises a proximal end 112 configured to mate with an intravascular access apparatus 100, a distal end 114 configured to mate with a syringe 108, a hollow, central chamber 116 between the proximal end and the distal end, and a plurality of blood testing devices 10 according to the present invention, each blood testing device 10 joined to the hollow, central chamber 116. In a preferred embodiment, the distal end 114 is configured to mate with the proximal end of a standard flash plug or the proximal end of a syringe 108 (as shown). This embodiment allows blood from one source to be tested using a plurality of blood testing devices 10 according to the present invention, as will be understood by those with skill in the art with reference to this disclosure.

According to another embodiment, referring now to FIG. 11, blood testing device 10 takes the place of a standard flash plug, the proximal end 12 of blood testing device 10 is configured to mate with the introducer part 102 of a intravascular access apparatus 100, as shown in FIG. 11, and additionally optionally the distal end 14 of blood testing device 10 may be configured to mate (not shown) integrally or removably with a blood collection device such as syringe 108.

According to another embodiment of the present invention, there is provided a method for testing blood for the presence or for the amount of various constituents, such as glucose, cardiac enzymes or red cells, or for testing blood to determine the level of various parameters, such as pH, or for testing blood for a combination of the preceding. The method comprises providing a blood testing device according to the present invention. The method additionally comprises providing a test strip, a test strip reader and a blood containing apparatus, such as an intravascular access apparatus or a syringe. The test strip can be any suitable test strip with a proximal end configured to be inserted into the distal end cap of the blood testing device. The test strip reader can be any suitable test strip reader configured to accept the distal end of the test strip and, after accepting the distal end, to provide the desired information on blood that has contacted the proximal end of the test strip. The blood containing apparatus can be any intravascular access apparatus suitable for accessing a vascular structure and suitable for joining to the proximal end of a blood testing device according to the present invention, or any other suitable blood containing apparatus as will be understood by those with skill in the art with reference to this disclosure.

The blood testing device can be provided with or without the test strip inserted through the distal end cap and into the distal portion and intermediate portion of the blood testing device. If the blood testing device is provided with the test strip inserted through the distal end cap and into the distal portion and intermediate portion of the blood testing device, then the combined blood testing device and test strip can be provided with the distal end of the test strip already inserted into the test strip reader. Further, the blood testing device, whether or not combined with the test strip or with the test strip and test strip reader together, can be provided having its proximal end already inserted into the distal end of the blood containing apparatus or can be provided without its proximal end already inserted into the distal end of the blood containing apparatus. As will be understood by those with skill in the art with reference to this disclosure, the blood containing apparatus, blood testing device, test strip and test strip reader are assembled together during the method of the present invention, and can be assembled in various orders. In a particularly preferred embodiment, the distal end of the test strip is inserted into the test strip reader, and then the proximal end of the test strip is inserted into the distal end cap of the blood testing device and through or around the inner membrane after blood has entered the intermediate portion of the blood testing device.

Next, a patient is selected who is to have his or her blood tested. If the blood containing apparatus is an intravascular access apparatus, the patient is prepped in a standard manner and intravascular access is begun using the intravascular access apparatus according to standard techniques, as will be understood by those with skill in the art with reference to this disclosure. If the proximal end of the blood testing device was not already inserted into the distal end of the intravascular access apparatus before obtaining intravascular access, then the proximal end of the blood testing device is inserted into the distal end of the intravascular access apparatus, such as by replacing the flash plug on the intravascular access apparatus with the proximal portion of the blood testing device. Preferably, the proximal end of the blood testing device is inserted into the distal end of the intravascular access apparatus before insertion of the intravascular access apparatus. Alternately, a blood testing device is provided according to the present invention, without a proximal portion and with the intermediate portion integrally affixed to the distal end of the introducer portion of a standard intravascular access apparatus.

Once vascular access is obtained and the blood testing device and the intravascular access apparatus are combined, blood from the patient's accessed vascular structure will pass through the one or more than one opening in the proximal end of the proximal portion into the hollow, central chamber of the proximal portion and, then, into the hollow, central chamber of the intermediate portion, or will pass directly into the hollow, central chamber of the intermediate portion if the blood testing device is integrally affixed to the intravascular access apparatus. Air that is present in the hollow, central chamber of the proximal portion, if present, and hollow, central chamber of the intermediate portion passes through the inner membrane into the hollow, central chamber of the distal portion and then, externally. If not already combined, the distal end of the test strip is inserted into the test strip reader and the proximal end of the test strip is inserted into the distal end cap, through the distal portion and into the intermediate portion of the blood testing device. The blood in the hollow, central chamber of the intermediate portion will, then, contact the proximal end of the test strip.

Next, the distal end of the test strip is inserted into an appropriate test strip reader, if not done previously. The operator uses information provided by the test strip reader, such as a digital readout or visual indicator, to determine the presence or the amount of one or more than one constituent of the blood, such as glucose, cardiac enzymes or red cells, or to determine the level of one or more than one parameter, such as pH, or to determine a combination of the preceding.

In one embodiment, the operator withdraws the blood testing device from the distal end of the intravascular access apparatus before using the test strip reader. In another embodiment, the blood testing device with an inserted test strip is removed from the intravascular access apparatus and an operator proceeds to establish intravascular access while the distal end of the test strip is being inserted into an appropriate testing apparatus, so that no time is being lost in establishing intravascular access while blood testing is being performed. In a preferred embodiment, the introducer part of the intravascular access apparatus with the blood testing device still inserted into the distal end are removed together, and an operator proceeds to establish intravascular access while the proximal end of the test strip is being inserted into distal end of the blood testing device, further increasing efficiency in establishing intravascular access while blood testing is being performed. In a particularly preferred embodiment, the blood testing device provided for use in this method is the embodiment shown in FIG. 5, and as will be understood by those with skill in the art with reference to this disclosure, removing the blood testing device necessarily removes the introducer part of the intravascular access apparatus from the vascular structure as the blood testing device is integrally affixed to the distal end of the introducer part of the intravascular access apparatus.

After the operator makes the appropriate determinations, the blood testing device with the test strip still inserted is removed from the test strip reader and, preferably with the introducer portion if being used with an introducer portion, is discarded in an appropriate manner as a biohazard. As can be appreciated by those with skill in the art with reference to this disclosure, the present method decreases the possibility of inaccurate blood test results due to test strip contamination, decreases the time necessary to analyze blood being tested from standard methods, decreases the risk of exposure of the operator to pathogens present in the blood, and simplifies disposal of the equipment used in testing the blood.

According to another embodiment of the present invention, there is provided a method for testing blood for the presence or for the amount of various constituents, such as glucose, cardiac enzymes or red cells, or for testing blood to determine the level of various parameters, such as pH, or for testing blood for a combination of the preceding. The method comprises providing a blood testing apparatus according to the present invention, where the blood testing apparatus comprises a plurality of blood testing devices according to the present invention joined to a common central chamber that is configured at the proximal end to combine removably or to be affixed with a blood containing apparatus, and to communicate with the blood containing part of the blood containing apparatus. As will be understood by those with skill in the art, with reference to this disclosure, the method corresponds to the method disclosed above, except that blood test strips can be inserted into the distal end caps of each blood testing device on the blood testing apparatus, expanding the capacity to test the blood.

According to another embodiment of the present invention, there is provided a method for testing blood for the presence or for the amount of various constituents, such as glucose, cardiac enzymes or red cells, or for testing blood to determine the level of various parameters, such as pH, or for testing blood for a combination of the preceding. The method comprises using a blood containing apparatus, such as a syringe, other than an intravascular access apparatus. In this method, blood is drawn into the blood containing apparatus other than by obtaining intravascular access directly through the distal end of the blood containing apparatus, such as by attaching the blood containing apparatus directly to an intravascular access apparatus or blood containing vial and drawing blood into the blood containing apparatus from that source.

Although the present invention has been discussed in considerable detail with reference to certain preferred embodiments, other embodiments are possible. Therefore, the scope of the appended claims should not be limited to the description of preferred embodiments contained in this disclosure.

1. A blood testing device for testing blood for the presence or for the amount of one or more than one constituent, or for testing blood to determine the level of one or more than one parameter, or for testing blood for a combination of the preceding, the blood testing device comprising:
a) a proximal end of the blood testing device and a distal end of the blood testing device;
b) an intermediate portion between the proximal end of the blood testing device and the distal end of the blood testing device, the proximal end being adapted to permit a flow of blood sample from an intravascular access apparatus to enter the blood testing device; and
c) a pressure compensation system to regulate [the] pressure in the intermediate portion; and
wherein the intermediate portion comprises a proximal end of the intermediate portion comprising one or more than one opening, a distal end of the intermediate portion and, between the proximal end of the intermediate portion and the distal end of the intermediate portion, comprises a hollow central chamber surrounded by an outer wall.

2. The blood testing device of claim 1, where the pressure compensation system comprises a distal portion between the proximal end of the blood testing device and the distal end of the blood testing device, an inner membrane separating the intermediate portion from the distal portion; and where the distal portion comprises a proximal end of the distal portion and a distal end of the distal portion and, between the proximal end of the distal portion and the distal end of the distal portion, comprises a hollow central chamber surrounded by an outer wall.

3. The blood testing device of claim 1, where the pressure compensation system comprises a pressure compensation valve.

4. The blood testing device of claim 3, where the pressure compensation valve is a duck bill valve.

5. The blood testing device of claim 2, further comprising a distal end cap at the distal end of the blood testing device comprising a first opening configured to receive a test strip.

6. The blood testing device of claim 1, where the outer wall of the intermediate portion is generally cylindrical.

7. The blood testing device of claim 2, where the outer wall of the distal portion is generally cylindrical.

8. The blood testing device of claim 1, further comprising a proximal portion between the proximal end of the blood testing device and the proximal end of the intermediate portion;
wherein the proximal portion comprises a hollow central chamber surrounded by an outer wall; and
wherein the proximal portion is configured to mate with a blood containing apparatus.

9. The blood testing device of claim 8, where the blood containing apparatus is selected from the group consisting of a syringe and the introducer part of an intravascular access apparatus.

10. The blood testing device of claim 8, where the outer wall of the proximal portion is generally cylindrical.

11. The blood testing device of claim 2, where the hollow central chamber of the intermediate portion and the hollow central chamber of the distal portion comprise generally rectangular inferior parts that communicate with each other and that are configured to accept a test strip.

13. The blood testing device of claim 1, where the outer wall of the intermediate portion is substantially transparent.

14. The blood testing device of claim 5, where the distal end cap is an integral part of the distal portion of the blood testing device.

15. The blood testing device of claim 5, where the distal end cap is removably affixed to the distal portion of the blood testing device.

16. The blood testing device of claim 5, where the distal end cap further comprises one or more than one second opening to allow the venting of air externally out of the distal portion of the blood testing device.

17. The blood testing device of claim 5, where the distal end cap is a semipermeable member.

18. The blood testing device of claim 5, where the distal end cap is a non-permeable member.

19. The blood testing device of claim 2, where the inner membrane is a semipermeable member.

20. The blood testing device of claim 2, where the inner membrane comprises one or more than one vent.

21. The blood testing device of claim 1, where the outer wall of the intermediate portion comprises one or more than one vent.

22. The blood testing device of claim 2, where the inner membrane comprises an opening configured to allow passage of a test strip from the distal portion of the blood testing device into the intermediate portion of the blood testing device.

23. The blood testing device of claim 1, where the intermediate portion is integrally affixed to an introducer portion of an intravascular access apparatus.

24. The blood testing device of claim 1, where the intermediate portion is configured to affix to an introducer portion of an intravascular access apparatus.

25. The blood testing device of claim 1, where the distal end is integrally affixed to a blood collection apparatus.

26. The blood testing device of claim 1, where the distal end is configured to affix to a blood collection apparatus.

27. The blood testing device of claim 23, where the distal end is integrally affixed to a blood collection apparatus.

28. The blood testing device of claim 23, where the distal end is configured to affix to a blood collection apparatus.

29. The blood testing device of claim 28, where the blood collection apparatus is a syringe.

30. The blood testing device of claim 24, where the distal end is integrally affixed to a blood collection apparatus.

31. The blood testing device of claim 24, where the distal end is configured to affix to a blood collection apparatus.

32. The blood testing device of claim 31, where the blood collection apparatus is a syringe.

33. A blood testing apparatus comprising a plurality of interconnected blood testing devices according to claim 1.

34. A method for testing blood for the presence or for the amount of one or more than one constituent, or for testing blood to determine the level of one or more than one parameter, or for testing blood for a combination of the preceding, the method comprising the steps of:
a) providing a blood testing device according to claim 1, a test strip comprising a proximal end and a distal end, a test strip reader configured to accept the distal end of the test strip and a blood containing apparatus;
b) affixing the blood testing device to the blood containing apparatus and allowing blood present in the blood containing apparatus to enter into the intermediate portion of the blood testing device;
c) inserting the distal end of the test strip into the test strip reader and the proximal end of the test strip into the intermediate portion of the blood testing device; and
d) using information provided by the test strip reader to determine the presence or the amount of one or more than one constituent of the blood, or to determine the level of one or more than one parameter, or to determine a combination of the preceding.

35. The method of claim 34, where the blood containing apparatus provided in step a) is selected from the group consisting of an intravascular access apparatus and a syringe.

36. The method of claim 34, further comprising selecting a patient whose blood is to be tested and intravascularly accessing the patient's blood with the blood containing apparatus.

37. The method of claim 34, where the blood testing device and blood containing apparatus are affixed integrally in step b).

38. The method of claim 34, further comprising the step of discarding the blood testing device after step c).

39. Means for testing blood for the presence or for the amount of one or more than one constituent, or for testing blood to determine the level of one or more than one parameter, or for testing blood for a combination of the preceding, the means comprising:
a) a proximal end of the blood testing device and a distal end;
b) an intermediate portion between the proximal end and the distal end;
c) end means to receive a test strip; and
d) means to compensate for pressure changes within the intermediate portion; and
wherein the intermediate portion comprises a proximal end of the intermediate portion comprising one or more than one opening, a distal end of the intermediate portion and, between the proximal end of the intermediate portion and the distal end of the intermediate portion, comprises a hollow central chamber surrounded by an outer wall.

40. A method for testing blood for the presence or for the amount of one or more than one constituent, or for testing blood to determine the level of one or more than one parameter, or for testing blood for a combination of the preceding, the method comprising the steps of:
a) providing a blood testing means, a test strip comprising a proximal end and a distal end, a test strip reader configured to accept the distal end of the test strip and a blood containing apparatus;
b) affixing the blood testing means to the blood containing apparatus and allowing blood present in the blood containing apparatus to enter into an intermediate portion of the blood testing means via a proximal portion of the blood testing means, which has at least one inlet opening;
c) inserting the proximal end of the test strip into the intermediate portion of the blood testing means and the distal end of the test strip into the test strip reader, and
d) using information provided by the test strip reader to determine the presence or the amount of one or more than one constituent of the blood, or to determine the level of one or more than one parameter, or to determine a combination of the preceding.
